(19) United States
(12) Patent Application Publication
(10) Pub. No.: US 2024/0000294 A1
(43) Pub. Date: Jan. 4, 2024

(54) ENDOSCOPE SYSTEM AND METHOD FOR OPERATING THE ENDOSCOPE SYSTEM

(71) Applicant: Qioptiq Photonics GmbH & Co. KG, Goettingen (DE)

(72) Inventor: Helmut Rauscher, Goettingen (DE)

(73) Assignee: Qioptiq Photonics GmbH & Co. KG, Goettingen (DE)

(21) Appl. No.: 18/362,588

(22) Filed: Jul. 31, 2023

Related U.S. Application Data
(63) Continuation of application No. PCT/US22/14832, filed on Feb. 2, 2022.
(60) Provisional application No. 63/145,587, filed on Feb. 4, 2021.

(51) Int. Cl. A61B 1/00; A61B 1/04

(52) U.S. Cl. CPC A61B 1/0016; A61B 1/00066; A61B 1/00045; A61B 1/04; A61B 1/00193; A61B 1/00165

(57) ABSTRACT

An endoscope system for imaging a sample, an inner part of a patient, or an organ with an imaging device includes an endoscope tube with a proximal end and a distal end configured to mount the imaging device. A handle at the endoscope tube proximal end is configured to move the endoscope tube rotationally. An interface is configured to removably attach the endoscope tube to the handle and to rotate the endoscope tube around a main axis relative to the handle without restriction.

### CROSS-REFERENCE TO RELATED APPLICATIONS

This application claims the benefit of U.S. Provisional Patent Application Ser. No. 63/145,587, filed Feb. 4, 2021, entitled “Endoscope System and Method for Operating the Endoscope System,” which is incorporated by reference herein in its entirety.

### FIELD OF THE INVENTION

The present invention relates to an endoscope system and a method for operating the endoscope system.

### BACKGROUND OF THE INVENTION

The demands placed on medical devices for improved performance are constantly increasing. One requirement is to improve the operability of medical devices. Very strict safety requirements must be met by medical devices, in particular, medical devices that are in direct contact with a patient. Particularly, endoscopes are subject to high safety and temperature requirements, and the endoscopes preferably provide improved images and are as easy as possible for a surgeon to operate.

Typically, endoscopes do not have a point of separation between the endoscope tube and its holder. Further, typically endoscopes are completely fixed systems end-to-end. Rotating such endoscopes around its main axis results in twisting and potentially damaging fixed internal cables. This limits the maneuverability and hence field-of-view of the optical devices of the endoscope. Further, cleaning of this type of endoscope is problematic, as generally they can only be cleaned by wiping with a germicide, as autoclaving is not possible because they are not waterproof throughout. Therefore, there is a need for an endoscope system providing improved usage and safety.

### SUMMARY OF THE INVENTION

Embodiments of the present invention provide an endoscope system and method for operating the endoscope system. Briefly described, the present disclosure relates to an endoscope system for imaging a sample, an inner part of a patient, or an organ. An imaging device includes an endoscope tube with a proximal end and a distal end configured to mount the imaging device. A handle at the endoscope tube proximal end is configured to move the endoscope tube rotationally. An interface is configured to removably attach the endoscope tube to the handle and to rotate the endoscope tube around a main axis relative to the handle without restriction.

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principals of the invention.

FIG. 1A is a schematic diagram of an exemplary first embodiment of an endoscope system.

FIG. 1B is a schematic drawing detailing a view of a section around interfaces of FIG. 1A.

FIG. 2A is a schematic drawing showing a cross-sectional view of an interface of FIG. 1A.

FIG. 2B is a schematic drawing showing an exploded view of the interface of FIG. 2A.

FIG. 3 is a schematic drawing showing a pivot bearing as a part of the interface of FIG. 1A.

FIG. 4 is a schematic drawing showing a rotary motor providing an inner portion of the interface of FIG. 1A.

FIG. 5 is a schematic drawing showing a sectional view of a detail of the interface of FIG. 1A.

FIG. 6 is a schematic drawing of a view of a cross-sectional plane along line A of FIG. 5.

FIG. 7A is a schematic drawing showing optical and electromagnetic connections of the interface of FIG. 1A.

FIG. 7B is a schematic drawing showing a cross-section of optical and electromagnetic connections of FIG. 7A.

FIG. 8 is a schematic drawing showing a drive for a rotation of the endoscope tube of FIG. 1A.

FIG. 9 is a schematic drawing showing coupling light sources under the embodiment of FIG. 1A.

FIG. 10A is a schematic drawing showing a detail of the handle and the endoscope tube of FIG. 1A.

FIG. 10B is a schematic drawing showing a clamping device as a part of the interface of FIG. 1A.

FIG. 10C is a schematic drawing showing a coupling between the handle and the endoscope tube of FIG. 1A.

### DETAILED DESCRIPTION

The following definitions are useful for interpreting terms applied to features of the embodiments disclosed herein, and are meant only to define elements within the disclosure.

As used herein, the expression “endoscope system” may describe a device, in particular a medical device for imaging a sample, an inner part of a patient, or an organ. The endoscope system typically includes at least an endoscope tube, and may provide images from an inner part of a patient. The endoscope system may further include an interface, and in particular, a galvanic isolation interface. The galvanic isolation interface may apply, for example, a high voltage on the order of 4000 V and induce a current flow on the order of 10 μA or less so that a patient is protected during examination.

As used herein, the expression “endoscope tube” may describe a part of the endoscope system, which is adapted to be at least partially inserted within a patient. In particular, the endoscope tube has a distal end that is inserted into the patient. A camera disposed at the distal end of the endoscope tube includes at least one image sensor and/or an objective that provides internal images of the patient. A proximal end of the endoscope tube is opposite the distal end is generally not inserted into the patient. The endoscope tube may be rigid, or alternatively be flexible. The expression “main axis”, in particular “main axis of the endoscope tube” refers to an axis of the largest extension of the endoscope tube, i.e., usually a longitudinal direction of the endoscope tube.

As used herein, the term “interface” refers to a connection between two devices, for example an endoscope tube and a handle, wherein different signals or energy of different types may be transmitted between the two connected devices via the interface. In particular, the interface transmits one or more of analog or digital electrical data, digital optical data, electric, and or optical energy (light). The interface may be rotationally symmetric with respect to the main axis. The interface may be described in a functional way, such as including any type of guiding signals and/or energy from the endoscope tube to a handle; e.g., the interface may have any kind of capacitive, inductive and/or electrical coupling, as well as a functionality including as a transmitter and/or receiver of electrical and/or optical data. The interface may be adapted to transmit image data coming from the distal end of the endoscope tube and for receiving control data from a base unit at the endoscope tube proximal end. The interface may have a transmitter and/or a receiver chip accommodating short range and high bandwidth transmission of image data described in terms of resolution and refresh rate of a screen.

As used herein, the term “handle” refers to a mechanical part that enables to positioning or moving the endoscope tube into and/or inside the patient. The handle may couple rigidly to the endoscope tube with respect to the main axis, so that the handle may determine a direction of the endoscope tube along a longitudinal position of the endoscope tube. An operator, a surgeon for example, may manually operate the handle. Alternatively, the handle may be operated automatically by a robot arm, or the handle may be appropriately operated both manually and robotically. The handle may allow for any rotational movement of the endoscope tube around its main axis while supported by the interface being connected between the handle and the endoscope tube.

As used herein, the expression “inner rotational degree of freedom” refers to a property of the interface enabling an endless, unhindered turning of the endoscope tube in both directions, clockwise and counterclockwise, relative to a rotational axis of a handle. A rotation of 360 degree may create a state which equals a non-rotated state of 0 degree. After a full 360-degree rotation, the orientation of the endoscope tube relative to the handle may be of the original state or initial state. After any rotation of the endoscope tube supported by the interface relative to the handle, the endoscope system may be free of any permanent or non-permanent internal twisting. The interface may have at least partially rotational symmetry to support this movement. At an inner conjunction, the interface may have two rotationally symmetric parts, a first part of which may rigidly couple to the endoscope tube and a second part may rigidly couple to the handle. The interface may be seen as an integrative part of the endoscope tube and/or of the handle and at least as a part of the endo scope system. The interface has the mechanical property of an inner unhindered rotatability and an electrical property of providing an information and/or energy flow or exchange between the endoscope tube and the handle.

As used within this disclosure, the term “conduit” refers to a means to conduct information and/or energy, for example, electrical wiring or an optical waveguide.

In exemplary embodiments of an endoscope system described below, the interface is accessible and operable such that the endoscope tube and the handle may be connected and disconnected. Within this disclosure, “connectable and disconnectable” or “couplable or decouplable” indicates the interface may be mechanically operated and/or may provide electrical and/or optical connectivity when in the connected state. In the connected state the interface may allow for rotating the endoscope tube relative to the handle, and may allow for transmitting electric and/or optical data and/or energy. Further, the endoscope tube and the handle may be disconnected from each other so that the endoscope tube and the handle are free of any mechanical and/or electrical and/or optical connection. While disconnected the endoscope tube may be cleaned and maintained separately from the handle and other parts of the endoscope system.

According to the exemplary embodiment of the endoscope system described below, the interface incorporates a galvanic isolation so that the endoscope tube and the handle are galvanically isolated when being connected to each other by the interface. As used herein, the expression “galvanic isolated” or “galvanic isolation” may describe a property of the interface providing electrical isolation, for example across the interface coupling the endoscope tube and the handle. In particular, the galvanic isolation interface may provide a galvanic isolation when the endoscope tube is coupled with its proximal end towards the handle. If the interface connects the endoscope tube and the handle there may be tools to support this coupling. Alternatively, the interface may allow for manually coupling and/or decoupling of the endoscope tube and the handle.

Regarding the interface, as used within this disclosure “freely accessible” refers to providing manual or tool supported coupling and/or decoupling the interface.

As used herein the expression “offset angle” refers to an angular deviation from a reference axis. For example, offset angle may describe an angle spanned between a camera direction (in which direction the camera points to take images) and the main axis of the endoscope tube. For example, an offset angle may be 30 degrees, i.e., that the camera points in a different direction compared with the endoscope tube. However, the offset angle may be negligible or zero (0 degrees), as an inner rotation of the interface may merely cause a different orientation of the images which are taken. In particular, the offset angle may be provided and adapted by an objective being a part of the camera.

Reference will now be made in detail to embodiments of the present invention, examples of which are illustrated in the accompanying drawings. Wherever possible, the same reference numbers are used in the drawings and the description to refer to the same or like parts.

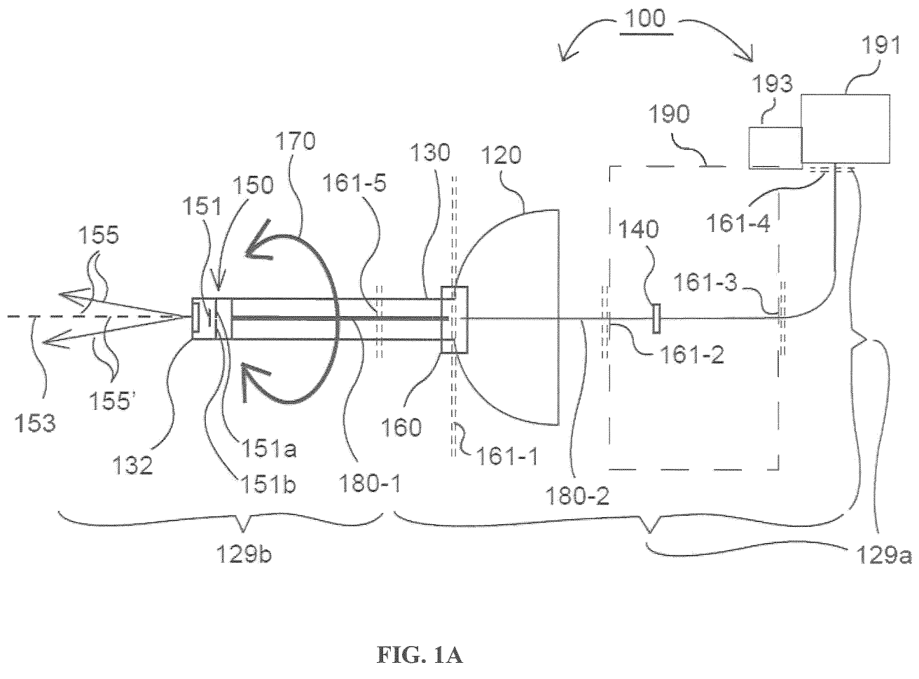

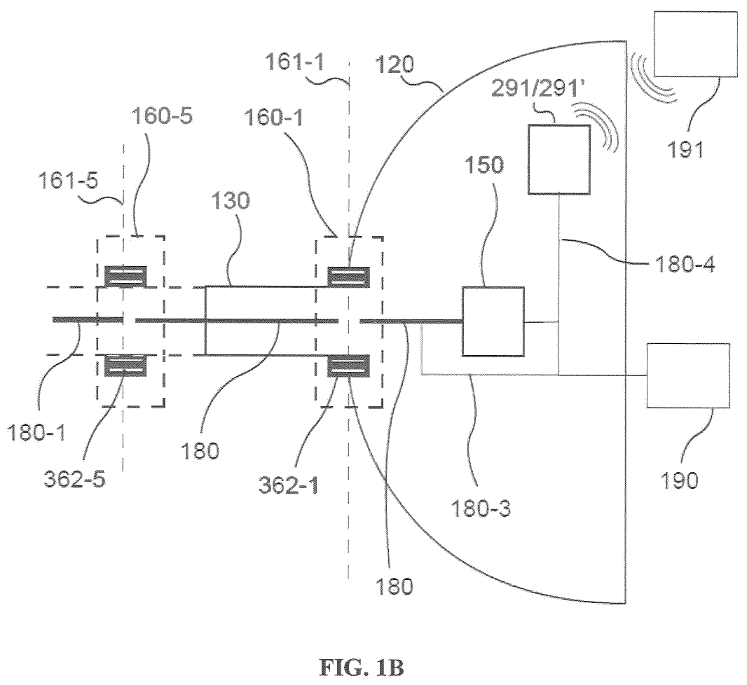

FIG. 1A shows a first embodiment of an endoscope system 100 that includes an endoscope tube 130 adapted to capture images from the inner part of a patient, a handle 120 for moving the endoscope tube 130, and an interface 160 arranged between the endoscope tube 130 and the handle 120. The interface 160 provides a rotational degree of freedom, so that the endoscope tube is rotatable about its main axis 153, and also rotatable relative to the handle 120.

In order to capture images, a camera unit 150 having a single image sensor 151 is disposed at a distal end 132 of the endoscope tube 130. Alternatively, or additionally to the single image sensor 151, the camera unit 150 may include an image sensor pair including a first image sensor 151a and a second image sensor 151b for capturing stereo images. The camera unit 150 may capture images with a camera angle having an offset angle 155 relative to the main axis 153 of the endoscope tube 130. Similar, first and second image sensors 151a, 151b may capture images with an offset angle 155′ relative to the main axis 153 of the endoscope tube 130. By a rotation 170 of the endoscope tube 130 a camera perspective may vary showing a wider area if the offset angles 155, or 155′ are greater than 0 degrees. If the offset angles 155, 155′ are 0 degrees only an orientation change of the same area may be captured by the camera unit 150.

A transmission cable 180 (FIG. 1B) may transmit electrical information, electrical energy, optical information, and/or optical energy. The transmission cable 180 may be disposed between the endoscope tube 130 and the interface 160, and/or the transmission cable 180 may be disposed between the interface 160 and the handle 120. Under the first embodiment, the endoscope system 100 includes a display screen 191 for displaying images or videos conveyed from the image sensor 151, a base unit 190, and a proximal transmission cable part 180-2 for transmitting data and/or energy towards the display screen 191 and/or towards the endoscope tube 130. The base unit 190 may include a light source 140 to illuminate a region of the patient to be examined. A front end of the proximal transmission cable part 180-2 may couple to the interface 160 which in turn couples to the distal transmission cable part 180-1. Herein, the distal transmission cable part 180-1 and the proximal transmission cable part 180-2 are together referred to as the expression transmission cable 180 (FIG. 1B).

As shown by FIG. 1A the interface 160 may be located in a plurality of positions. The interface 160 may be located at a first interface location 161-1 between the endoscope tube 130 and the handle 120, a second interface location 161-2 between the handle 120 and the base unit 190, a third interface location 161-3 between the base unit 190 and the display screen 191 close to the base unit 190, and a fourth interface location 161-4 between the base unit 190 and the display screen 191 but closer to the display screen 191. The interface 160 may also be located at a fifth interface location 161-5 at the endoscope tube 130 close to the handle 120. The fifth interface location 161-5 indicates a special separation: a first portion oriented toward the endoscope tube 130 may be denoted as surgical section 129b that may contact the patient. A second towards the display screen 191 including all five interface locations 161-1,-2,-3,-4,-5 may be denoted as operating section 129a, the part of the endoscope system 100 directly operated by the surgeon. A power supply connection 193 may be connected to the display screen 191 and/or to the base unit 190.

The endoscope tube 130 is made of a material that is biocompatible, and preferably an electrically conducting material for shielding the inner electronics from electromagnetic interference (EMI), such as steel and/or titanium, among others.

FIG. 1B is a schematic view showing a section around the interface 160 and the interface 160-5. The two interfaces 160-1 and 160-5 may both include pivot bearings 362-1, 362-5 at the first position 160-1, and at the fifth position 160-5, respectively. Data captured at the distal end 132 (FIG. 1A) of the endoscope tube 130 may be transmitted via the transmission cable 180 to the interface 160-1 here shown located at least partially inside of the handle 120. A control data cable 180-3 (being a portion of the transmission cable 180) may transmit control data from the base unit 190 to the interface 160. In a reverse direction, a transmission cable 180-4 may transmit data towards a transmitter/receiver 291, 291′ which sends image data towards the display screen 191 equipped to receive the respective image data directly. Alternatively, the image data may be transmitted to the display screen indirectly. For example, due to high bandwidth of video data a transmission in the 60 GHz band may be used, so the corresponding transmitted waves propagate like visible light and any object between transmitter and receiver can disturb the connection. To address this problem, two or more receivers may be situated in different locations, and a switch may be configured to select the best received signal and route this to the screen. There are many off-the-shelf devices on the market which use these 60 GHz technique to stream video content to a display screen wirelessly. For example, the ultra-fast 60 GHz wireless 4k UHD transmitters and receivers from IOGEAR. Furthermore, there are silicon chip-sets from Analog Devices Inc. for a smooth integration of this wireless interface in custom electronics designs available.

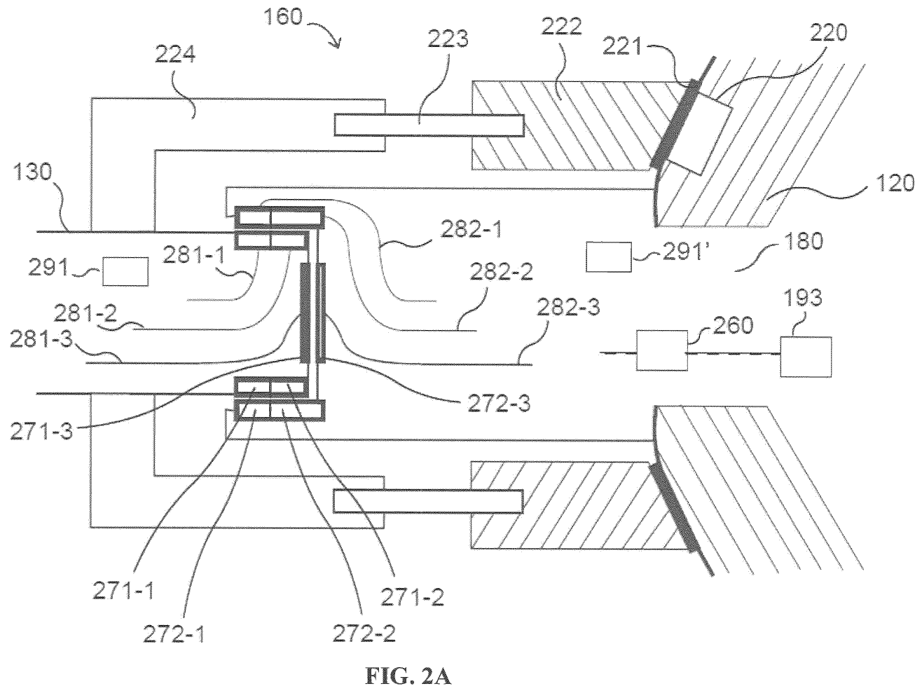

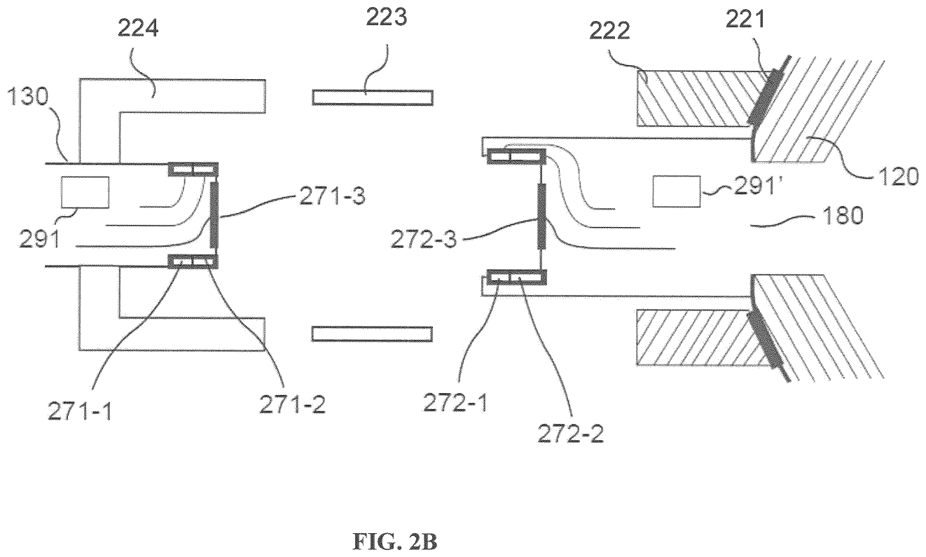

The endoscope system 100 may include a rotary motor 220 (FIG. 2A), so that an inner rotation of the galvanic isolation interface is caused by the rotary motor. The rotary motor 220 may be helpful for controlling and causing the inner rotation of the interface 160. In alternative embodiments the rotary motor 220 may be integrated into the endoscope tube 130, the interface 160 and/or the handle 120.

FIG. 2A shows a cross-sectional view of the interface 160 under the first embodiment. The handle 120 may be provided with the motor 220 coupling to a gear ring 222 via a gear 221. The gear ring 222 may engage with a transmission element 223 which then may engage with a flange 224 coupling to the endoscope tube 130, so that the motor 220 may drive a rotation of the endoscope tube 130. The transmission element 223 may have any appropriate form in order to transfer the power from the rotary motor 220 towards the endoscope tube 130, such as cylindrical or having transmission bars. In particular, the transmission element 223 may have a very low electrical conductivity so that no or negligible electricity from the rotary motor 220 may be transmitted to the endoscope tube 130 with this mechanical conjunction. For example, the transmission element 223 and the flange 224 may be made of an electrical isolating material such as Acrylnitril-Butadien-Styrol (ABS), polyvinyl chloride (PVC), or nylon, or another suitable material having a specific resistance of isolators on the order of 10¹³ Ohm*mm²/m or greater.

A first tube sided electrode ring 271-1 and a second tube sided electrode ring 271-2 may be an integral part of the endoscope tube 130, or the part of the interface 160 being assigned to the endoscope tube 130. Similarly, a first handle sided electrode ring 272-1 and a second handle sided electrode ring 272-2 may be an integral part of the handle 120, or the part of the interface 160 being assigned to the handle 120. For a data or electrical power transmission, the first tube sided electrode ring 271-1 may lie proximately inside the first handle sided electrode ring 272-1, but still having a gap in between, for a capacitive and/or inductive transmission of data and/or electrical power. For further safety and reliability all coupling elements (electrode rings, coils) may be covered by a thin electrical isolating material like ABS, PVC or so on, such that no electrically conductive part is exposed to the outside of the tube at the interface area.

Analogously, the second tube sided electrode ring 271-2 may lie directly inside a second handle sided electrode ring 272-2 for a capacitive and/or inductive transmission of data and/or electrical power. The same may apply to a tube sided pad 271-3 and a handle sided pad 272-3 positioned opposite to each other with a gap therebetween, for transmitting data by a capacitive coupling. Each of the first tube sided electrode ring 271-1, the second tube sided electrode ring 271-2, the first handle sided electrode ring 272-1, and the second handle sided electrode ring 272-2 may couple to a first tube sided cable 281-1, a second tube sided cable 281-2, a first handle sided cable 282-1, and a second handle sided cable 282-2, respectively Alike, the tube sided electrode pad 271-3, and the handle sided electrode pad 272-3, may be connected to a third tube sided cable 281-3, and a third handle sided cable 282-3, wherein the tube sided cables connect to the endoscope tube 130 and the handle sided cables connect to the handle 120.

For further safety, a rigid galvanic isolation 260 may connect between a power supply connection 193 and the transmission cable 180. Preferably, a transmitter/receiver combination 291,291′ may be arranged within or close to the endoscope tube 130 (preferably the transmitter 291) and/or inside or close to the handle 120 (preferably the receiver 291′). Using two video channels for stereo imaging corresponds to two transmitters inside 130 and two receivers inside 120. In this case the distance of the two transmitter antennas is greater than the distances between the transmitter and associated receiver antennas, for example, by at least an order of magnitude, for example a distance of 2 mm between the transmitter and the associated receiver antennas and a distance of 20 mm between the two transmitter antennas inside 130. This arrangement provides a good coexistence of the two transmission channels without disturbing one another. The combination of the transmitter 291 and the receiver 291′ may provide additional or supplementary data exchange.

FIG. 2B shows an exploded view of the interface 160. From the exploded view it becomes apparent that the electrical parts (electrode rings, electrode pads, cables) assigned to the endoscope tube 130 stay with the endoscope tube 130 whereas the electrical parts assigned to the handle 120 stay with the handle 120. Thus, a gap of about 0.1 mm to 0.5 mm and at least one non-conducting surface (preferably on the handle 120) providing a galvanic isolation may be formed between the endoscope tube 130 and the handle 120. For example, the galvanic isolation may be configured to withstand a voltage of at least 4 kV at a current flow of less than 1 mA. The material forming the isolating surface may have, for example, a dielectric strength of at least 20 kV/mm, such as ABS. The flange 224 may be assigned appropriately towards the endoscope tube 130 or the handle 120. By this separation, the interface 160 may allow for coupling the endoscope tube 130 to and decoupling the endoscope tube 130 from the handle 120, enabling rotation of the endoscope tube 130 relative to the handle 120.

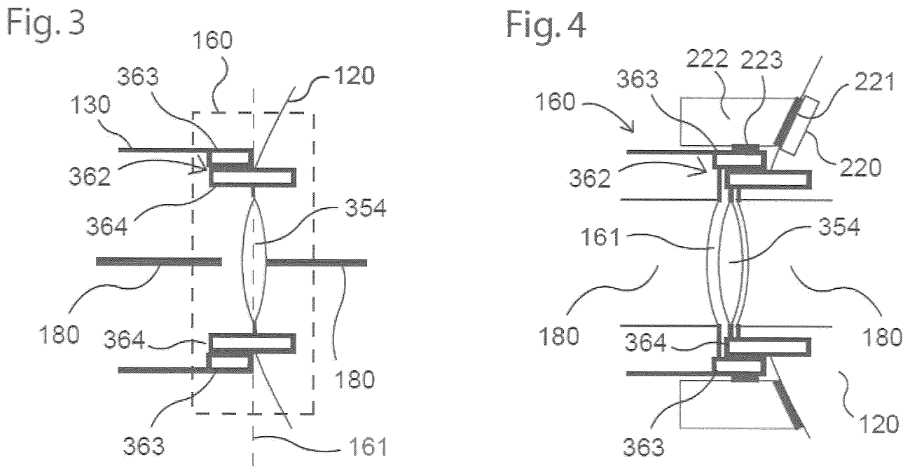

FIG. 3 illustrates a pivot bearing 362 as a part of an interface 160, the pivot bearings 362-1, 362-5 also shown in FIG. 1B. In general, the pivot bearing 362 may include a bearing outer ring 363 assigned to the endoscope tube 130, whereas a bearing inner ring 364 is assigned to the handle 120. An interface lens 354 may be assigned and fixedly coupled to the handle 120 and may form an optical connection between the handle 120 and the endoscope tube 130.

FIG. 4 illustrates a rotary motor 220 providing an alternative inner rotation when compared to the rotary motor 220 in FIG. 2A. Here, the transmission element 223 may directly engage with the bearing outer ring 363 instead with a flange 224 (see FIG. 2A).

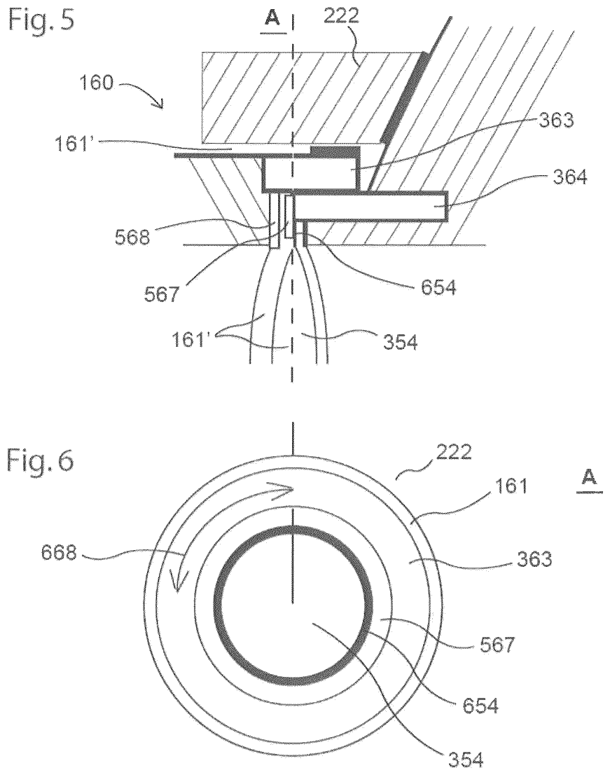

FIG. 5 is a sectional view of a detail of the interface 160 shown in FIG. 4. FIG. 5 shows a gap 161′ providing a galvanic isolation (and describing a way to functionally locate the position of the interface 160). A lens suspension 654 assigned to the handle 120 may fixedly be coupled between the handle 120 and the interface lens 354, so that the interface lens 354 is an integral part of the handle 120. In the interface 160 shown by FIG. 5 a data and/or energy transmission may be provided by a handle sided electrode O-ring 567 and a tube sided electrode O-ring 568 which lie opposite one another. In FIG. 5 the letter A indicates a cross-section shown by FIG. 6.

FIG. 6 is a view of a cross-sectional plane along A as indicated in FIG. 5. Proceeding inward from the outside, the gear ring 222 is surrounds the gap 161′, providing a galvanic isolation between the gear ring 222 (of the handle 120) and the bearing outer ring 363 (of the endoscope tube 130). The bearing outer ring 363 is preferably made of an electrical insulating material to provide electrical separation to the lens suspension 654, wherein the central part is the interface lens 354. A rotation 668 indicates that the integral parts of the endoscope tube 130 (not all shown here) rotate mutually relative to the integral parts of the handle 120.

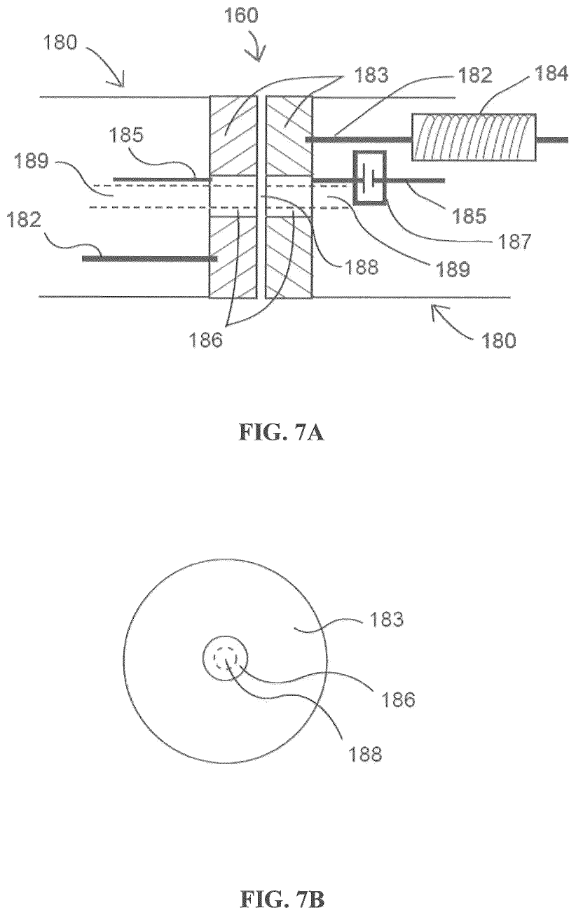

FIG. 7A schematically depicts optical and electromagnetic connections. In general, the interface 160 provides a galvanic isolated connection for a power cable 182 with a galvanically separated power coupling 183. The galvanically separated power coupling 183 may correspond to an inductive coupling 184. Further, a data cable 185 may be galvanically coupled with a capacitive coupling 187. Finally, an optical data and/or light cable 189 may be coupled by an optical coupling 188. Any of the cables, including the power cable 182, the galvanically separated power coupling 183, the data cable 185, and the optical data and/or light cable 189 may be regarded as an integral part of the transmission cable 180.

An inductive coupling takes place between two conductor coils which are located near to each other by the magnetic field. Electrical power, for example, up to 20 W, may be transferred from the transmitter coil to the receiver coil even if an electric isolator (plastics) is located between the two coils. Therefor inductive coupling may be preferable to transfer electrical energy and electrical signals, for example, with a bandwidth of up to 10 MHz. The coils for the inductive coupling may be formed as windings of a wire or as flat coils on a PCB. The coils may lie side by side or concentric to each other.

A capacitive coupling takes place between two electrically conductive areas located adjacent to each other. The grade of coupling increases if the distance between the two areas decreases and/or a dielectric medium is located between the two areas. Because an isolating plastic is a better dielectric medium than air because of its higher relative permittivity, the plastic insulation barrier formed on the surface of 130 increases the capacitive coupling, for example, by about a factor of 4 for equal area separations. Because of the small resulting capacitances, for example, not more then 10 pF, the capacitive coupling is preferable for high frequency signals like the video data or other high frequency communication, for example, having signal frequencies of 100 MHz and higher.

An optical coupling takes place between two light guides through a free air gap. A large amount of light can be coupled from a light source by a bundle of glass fibers over an interface (free air gap) to a light guide inside of the endoscope tube 130. In addition, modulated light may be used to transfer the high frequency video data over a light guiding ring (part of the endoscopic tube 130) to a glass fiber (part of the handle 120 and/or the proximal transmission cable part 180-2) and to an optical receiver inside the handle 120 and/or the base unit 190.

FIG. 7B illustrates a cross-section view of the optical and electromagnetic connections shown in FIG. 7A. The galvanically separated power coupling 183 for the power cable 182 may require the largest extension. A ring area is formed from the outer diameter to the half of the outer diameter. Wherein the galvanically separated data coupling 186 and the optical coupling 188 being the most inner part of the rotation symmetrical interface 160 (not shown) may include the inner remaining circle area which may be separated into two about equal areas for the optical coupling 188 and the data coupling 186.

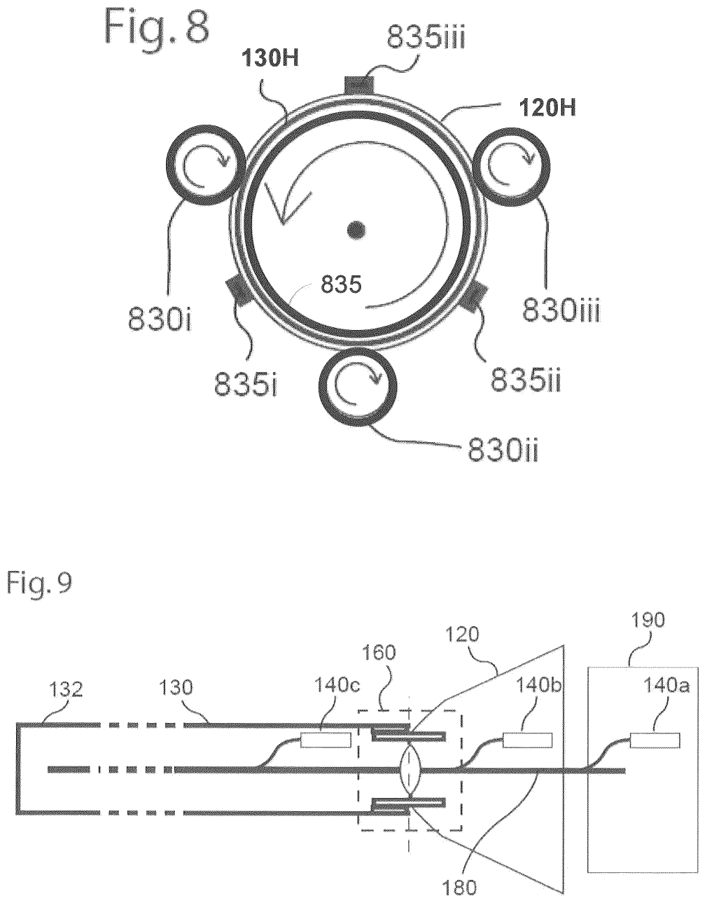

FIG. 8 illustrates a drive for a rotation of the endoscope tube 130H, (shown in cross-section) the drive having a plurality of friction rollers 830i, 830ii, 830iii evenly spaced around the endoscope tube proximal end, for example, three friction rollers arranged at an angle of 120°. Each of the plurality of friction rollers 830i, 830ii, 830iii passes through an opening in and end portion of the handle 120 so that each friction roller 830i, 830ii, 830iii is in direct physical contact with the endoscope tube 130H. A common rotation of the three friction rollers 830i, 830ii, 830iii in one direction causes a rotation of the endoscope tube 130H in the opposite direction. The three friction rollers 830i, 830ii, 830iii may consist of an electrically insulating material or be covered by an electrically insulating material, for example galvanized rubber, so that the endoscope tube 130H is electrically isolated against the three friction rollers 183i, 183ii, 183iii. As an alternative or supplementary, the endoscope tube 130H may include an electrically insulating material at least at its surface, so that the electrical insulation is achieved by this.

The three friction rollers 830i, 830ii, 830iii may additionally provide a rigid suspension of the endoscope tube 130H so that only a rotation of the endoscope tube 130H may happen, and a movement of the endoscope tube 130H in a radial direction is prevented. In addition to the drive for a rotation, a rotation angle detection device may include a permanent magnet 835 and hall sensors 835i, 835ii, 835iii so that the rotational state may be detected, and the detection of the rotational state may be a basis for controlling the friction rollers 830i, 830ii, 830iii. The hall sensors (or other relative or absolute positioning sensors) 835i, 835ii, 835iii may be located outside of the endoscope tube 160H wherein the permanent magnet 835 may be located on the hollow inside of the endoscope tube 160H and opposite to the hall sensors 835i, 835ii, 835iii. In particular, the permanent magnet 835 may resist higher temperatures up to 150° C. in order to maintain its function after a process of sterilization of the endoscope tube 130. For example, the permanent magnet may be a Samarium-Cobalt (SmCo) magnet.

FIG. 9 is a schematic view of a mechanism for coupling light sources 140a, 140b, 140c into the transmission cable 180. In particular, the first light source 140a may be located inside the base unit 190, the second light source 140b may be located inside the handle 120, and the third light source 140c may be located within the endoscope tube 130. All three light sources, the first light source 140a, the second light source 140b, and the third light source 140c couple the generated light into the transmission cable 180 which then guides the light towards the distal end 132 of the endoscope tube 130 for the use of illumination of the area of the patient to be examined. Electrical power for the third light source 140c may be provided via the interface 160, wherein the interface 160 may transmit the electrical power by an inductive coupling.

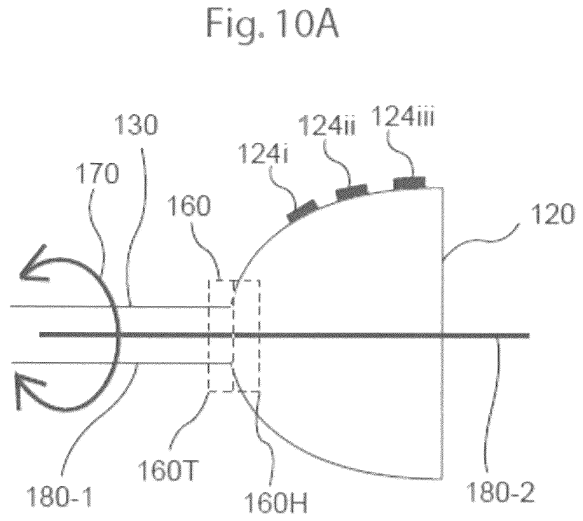

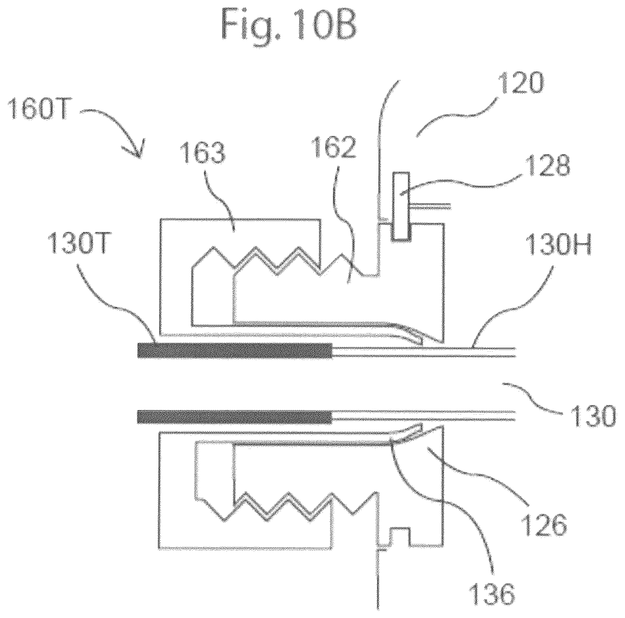

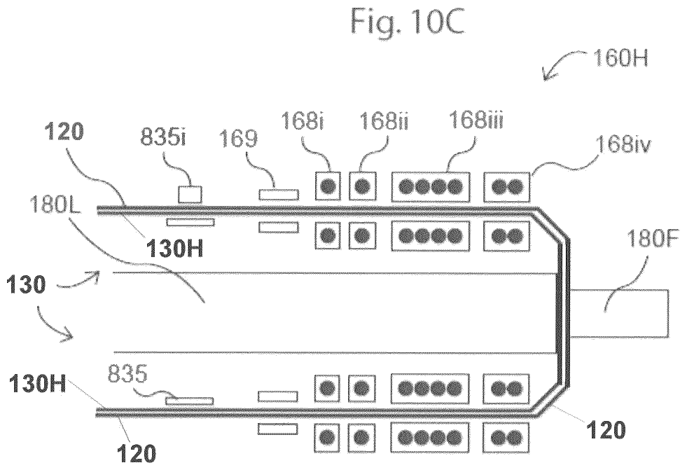

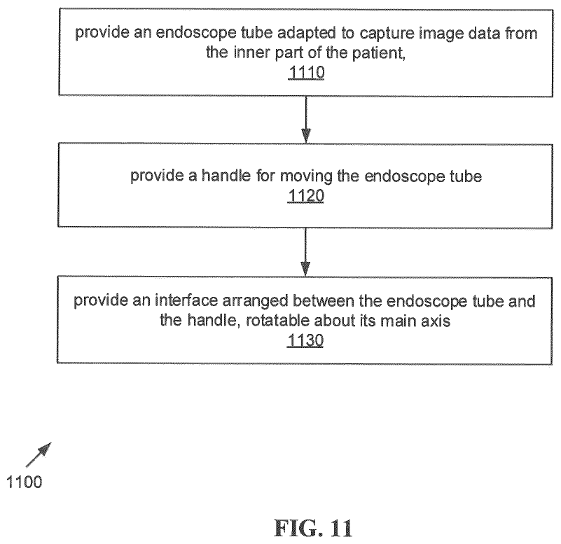

The interface 160 may include a clamping device by which the endoscope tube 130 is fixed to the handle 120 so that a motor providing a rotation can cause a rotation of the endoscope tube 130. FIG. 10A shows a detail of the handle 120 and the endoscope tube 130 where the handle 120 includes controls 124i, 124ii, 124iii, for example electrical actuators enabling the surgeon to control the movement of the endoscope tube 130. Further, a first rectangle with dotted lines indicate a clamping device 160T as a part of the interface 160, and a second rectangle with dotted lines indicates an electrical-optical coupling 160H being also a part of the interface 160. In particular, both the clamping device 160T and the electrical-optical coupling 160H may be arranged at least partially inside the handle 120. In particular, the clamping device 160T may be an integral part of the handle 120 and the electrical-optical coupling 160H may be located inside the handle 120. The clamping device 160T may be arranged towards the distal end of the endoscope tube 130, and the electrical-optical coupling 160H may be arranged towards the opposite, proximal end of the endoscope system 100 (see FIG. 1), such that the clamping devices 160T and the electrical-optical coupling 160H are rigidly coupled by the endoscope tube 130.

FIG. 10B shows a clamping device 160T, which only allows a rotational movement of the tube 130 if it is fastened, as a part of the interface 160 (see FIG. 10A). Under the first embodiment, the clamping device 160T includes a clamp screw 163 having a clamping element 136 which surrounds the endoscope tube 130 where the endoscope tube 130 includes an electrically insulating portion 130H. Further, the clamping device 160T, may have a clamping nut 162 with a slope portion 126, which may include an electrically insulating surface. By fastening the clamp screw 163 on the clamping nut 162, the clamping element 136 may interact with the slope portion 126 so that the clamping element 136 narrows towards the endoscope tube 130 and to clamp the endoscope tube 130. The endoscope tube 130 may include the insulating portion 130H and a metal portion 130T, the metal portion 130T extending towards the distal end of the endoscope tube 130.

A gear 128 is arranged with the handle 120 may mate with the clamping nut 162, so that a rotation of the gear 128 causes a rotation of the clamping nut 162, which in turn causes a rotation of the clamp screw 163, which further causes a rotation of the clamped endoscope tube 130.

A glass fiber optic bundle may be coupled with a light source providing light for illumination the specimen to be examined. The light source may be arranged in the handle 120 or towards a proximal end of the endoscope system 100. A glass fiber optic bundle or any bundle with a plurality of singular fiber cables for conducting light conveys light from the light source to a target to be illuminated. A light conductor, in particular a light transmission bar, may be coupled to the glass fiber optic bundle. The glass fiber bundle may be arranged in a handle side of the interface 160 and the light conductor or light transmission bar may be arranged inside the endoscope tube 130.

FIG. 10C shows an electrical-optical coupling 160H between the handle 120 and the endoscope tube 130, or the insulating portion 130H of the endoscope tube 130, respectively, the electrical-optical coupling 160H being also regarded as a part of the interface 160. The insulating portion 130H of the endoscope tube 130 may extend into the handle 120 in which the electrical power and the optical power may be transmitted. A light conductor 180L, e.g., formed as a light transmission bar, centrally located in the endoscope tube 130 may couple to a fiber optic bundle 180F which is provided inside the handle 120 which receives a light beam is introduced by a light source (not shown).

Further, the electrical-optical coupling 160H may include pairs of coils 168i, 168ii, 168iii, 168iv. For each of the coil pairs 168i, 168ii, 168iii, 168iv one coil may be arranged inside the endoscope tube 130 and the other coil may be arranged directly opposite hereto on an inner surface of the handle 120. Here, a rotary movement of each coil (e.g., of the endoscope tube-sided coil) of the two coils of one coil pair 168i, 168ii, 168iii, 168iv may be independent to the other coil (e.g., here the coil inside the handle 120). Alternatively, a rotary movement of the coils inside of the endoscope tube 130 may be independent from the coils arranged to and directly inside the handle 120.

For the control communication channel, a bidirectional bitrate of about 800 kBit/s is desirable. For video data a bitrate up to 4 GBit/s in a single channel mode or up to 2 GBit/s in a dual channel mode is desirable. In a non-limiting example of an rx/tx coil pair, the diameter of the rx-coil may be about 10 mm, and the diameter of the tx coil may be about 12 mm. Preferably, the same rx/tx coil pair is used for both power transfer and bidirectional control communication, for example, to transfer until about 10 W electrical energy over the inductive interface. The wireless power transfer is generally in a frequency range of about 100 kHz-200 kHz, with coil inductances of about 5 μH-10 μH.

For example, a first and second coil pair 168i, 168ii may provide an inductive coupling in a frequency range of 50 MHz to 2 GHz for providing two channels of video data transmission (if two image sensors 151A, 151B are used, see FIG. 1A). Further, an inductive coupling may be provided at a frequency of about 125 kHz by a third coil pair 168iii for transmitting electrical power from the handle 120 to the endoscope tube 130. At a frequency of about 13.56 MHz a fourth coil pair 124iv may provide a channel for transmitting digital information between the handle 120 and the endoscope tube 130. In particular, the digital information may provide digital control data which may be exchanged via the fourth coil pair 168iv. Each coil of the coil pairs 168i, 168ii, 168iii, 168iv may be at least partially surrounded by a ferrite sheathing to avoid undesired crosstalk. A ferrite carbine with a high permeability can be used as a layer between the coils and the endoscope tube 130. This ferrite also forms the magnetic flux lines so that the coupling factor between rx and tx coils increases. Because the wireless power transfer inside the interface 160 uses an inductive principle, the rx and tx coils are not covered with an electrically conducting layer, as this would only produce eddy currents inside the tube walls. Therefore, a non conducting material must be used to cover the coils, for example ABS or Teflon.

As an alternative, or in addition, to the coil pair 168i, 168ii, a RF-antenna pair 169 is arranged similarly surrounding the inside of the endoscope tube 130, and the inside of the handle 120 in which the endoscope tube 130, and by this providing a contact free high frequency exchange at a frequency of 60 GHz for two video channels. The integration of a rotation angle detection system may include the permanent magnet 835 and the hall sensors 835i, 835ii, 835iii, as depicted by FIG. 8 and described previously.

Under the first embodiment, the interface 160 within the endoscope system 100 facilitates unrestricted rotation of the endoscope tube 130 relative to the handle 120. Consequentially, a surgeon may use this combination of the endoscope tube and the handle without regard to any previous or subsequent turnings of the endoscope tube relative to the handle. This may allow the surgeon to concentrate on operating the patient while receiving information, for example image data shown on a screen. The interface 160 protects the patient from possibly dangerous electrical energy by integration of galvanic isolation. Further, the interface 160 may provide a solution for separating the endoscope tube 130 and the handle 120. This facilitates cleaning and maintaining the endoscope tube 130 independently from the handle 120, so the endoscope tube 130 may be maintained and cleaned better and more frequently, as the endoscope tube 130 is at least partially in contact with the patient. Thus, a galvanic isolation outside of the patient may provide a safe and efficient use of the endoscope system 100.

The rotational state of the interface 160 and the (transversal and longitudinal) movement of the endoscope tube 130 may captured while the endoscope system 100 is being operated manually and/or robotically, as well as partially manually and robotically. A state of rotation of the endoscope tube 130 relative to the handle may be captured and/or transmitted to a processing unit so the generation of images may be based at least partially on information regarding the inner rotation of the interface 160.

As an alternative or in addition, a transmitter/receiver combination may be used to transmit (processed or non-processed) image data towards a screen or control data towards the endoscope tube, or camera unit comprising the image sensor(s). Data from the image sensors may be processed within the handle and/or within a base unit and/or within a computer connecting to the screen.

One or more light sources may provide electromagnetic waves of different wavelengths. The wavelengths may depend on the purpose such as using visible light for displaying images in the visible range, or such as monochromatic waves for using fluorescent effects. The light sources may be arranged outside the portion of the endoscope tube coming in contact with the patient. The evoked electromagnetic waves may be coupled to a light wave guide, such as a fiber glass cable by which the waves may be guided to the distal end of the endoscope tube. The electromagnetic waves may pass the interface in central location so that passing the interface is independent of the inner rotational state of the interface and the relative rotation of the endoscope tube and the handle. Depending on the energy consumption the at least one light source may be integrated within the base unit, the handle, and/or the endoscope tube. The light sources may couple the generated light into the transmission cable which then guides the light towards the distal end of the endoscope tube for the use of illumination of the area of the patient to be examined. Electrical power for the third light source being arranged within the endoscope tube, and preferably closer to the handle, may be provided via the rotatable interface, wherein the interface may transmit the electrical power by an inductive coupling.

FIG. 11 is a flowchart 1100 illustrating an exemplary embodiment of a method for providing images within a patient. It should be noted that any process descriptions or blocks in flowcharts should be understood as representing modules, segments, portions of code, or steps that include one or more instructions for implementing specific logical functions in the process, and alternative implementations are included within the scope of the present invention in which functions may be executed out of order from that shown or discussed, including substantially concurrently or in reverse order, depending on the functionality involved, as would be understood by those reasonably skilled in the art of the present invention.

An endoscope tube adapted to capture image data from the inner part of the patient is provided, as shown by block 1110. The endoscope system includes at least one image sensor for capturing image data with a user specified offset angle. A handle for moving the endoscope tube is provided, as shown by block 1120. An interface arranged between the endoscope tube and the handle, rotatable about its main axis is provided, as shown by block 1130. The endoscope tube may be rotated to capture images in directions according to the offset angle with respect to the main axis of the endoscope tube.

A base unit collects, analyzes, and processes the image data for display on a screen. The base unit may be an independent device, or may be integrated inside the handle and/or the screen.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. An endoscope system for imaging a sample, an inner part of a patient, or an organ with an imaging device, comprising:
an endoscope tube, comprising a proximal end and a distal end configured to mount the imaging device;
a handle disposed at a proximal end of the endoscope tube, the handle being configured to move the endoscope tube rotationally; and
an interface configured to removably attach the endoscope tube to the handle, and to rotate the endoscope tube around a main axis relative to the handle.

2. The endoscope system according to claim 1, wherein the interface comprises a galvanic isolation between the handle and the endoscope tube.

3. The endoscope system of claim 1, wherein:
the endoscope tube comprises a handle sided portion;
the handle comprises a clamping device restrict longitudinal motion of the endoscope tube; and
at least one of the handle sided portion and the clamping device comprises an electrically isolating material electrically isolating the clamping device and the handle sided portion.

4. The endoscope system of claim 1, further comprising a conduit configured to convey at least one of the group of electrical information, electrical energy, optical information, and optical energy between the endoscope tube and the handle.

5. The endoscope system of claim 4, wherein the conduit comprises a glass fiber optic bundle detachably coupled to a light transmission bar.

6. The endoscope system of claim 1, further comprising the imaging device comprising at least one image sensor configured to capture images with a first offset angle, so that following an inner rotation images can be taken in directions which deviate by the first offset angle around a direction of a main axis of the endoscope tube.

7. The endoscope system of claim 1, further comprising a imaging device comprising at least one pair of image sensors for capturing stereo images, the at least one pair of image sensors comprising a first image sensor and a second image sensor, wherein the first image sensor and the second image sensor are spaced from each other, wherein the at least one pair of image sensors captures stereo images with a second offset angle, so that following an inner rotation of the interface, stereo images can be taken and/or generated in directions which deviate by the second offset angle around a direction of a main axis of the endoscope tube.

8. The endoscope system of claim 1, further comprising a rotary motor configure to drive rotation of the interface.

9. The endoscope system of claim 1 further comprising:
a display screen; and
a transmission cable coupled to the display screen and configured to transmit image data to the display screen.

10. The endoscope system of claim 9, further comprising at least one of the group consisting of a data transmitter, and data receiver configured transmitting data from at least one of the group of the endoscope tube, the handle, and a control unit to at least one of the group of the display screen, the control unit, and the handle.

11. The endoscope system according to claim 1, further comprising a rigid galvanic interface, arranged between a power supply connection and the interface configured to provide an inner grade of rotational freedom.

12. A method for using an endoscope system for imaging a sample, an inner part of a patient, or an organ, comprising the steps of:
providing an endoscope tube, adapted to capture images from the inner part of the patient;
providing a handle configured to rotationally move the endoscope tube;
providing an interface arranged between the endoscope tube and the handle, wherein the interface comprises an inner rotational degree of freedom, so that the endoscope tube is rotatable about its main axis; and
rotating the endoscope tube relative to the handle.

13. The method according to claim 12, further comprising at least one image sensor for capturing images with a first offset angle, wherein the method further comprises the steps of:
capturing images by the at least one image sensor providing images with the first offset angle; and
rotating the endoscope tube so that following an inner rotation of a galvanic interface the images can be taken in directions which deviate by the first offset angle around a direction of a main axis of the endoscope tube.

14. The method according to claim 13, further comprising the steps of:
providing at least one pair of image sensors comprising a first image sensor and a second image sensor, wherein the first image sensor and the second image sensor are spaced from each other, and wherein the at least one pair of image sensors captures stereo images with a second offset angle:
capturing stereo images with the at least one pair of image sensors configured to provide the images with the second offset angle; and
rotating the endoscope tube so that following the inner rotation the stereo images are taken in directions which deviate by the first offset angle around a direction of a main axis of the endoscope tube.

15. The method according to claim 13, wherein the method further comprises the steps of:
generating the inner rotation of the interface manually and/or robotically; and
generating composite images, wherein a state of the inner rotation of the interface is a basis of generating the composite images, composite stereo images, videos and/or stereo videos.
